(19) United States
(12) Patent Application Publication
(10) Pub. No.: US 2002/0070354 A1
(43) Pub. Date: Jun. 13, 2002

(54) Manufacturing method of mask for electron beam proximity exposure and mask

(76) Inventors: Nobuo Shimazu, Machida-city (JP); Takao Utsumi, Watchung, NJ (US)

Correspondence Address: NIXON PEABODY, LLP, 8180 GREENSBORO DRIVE, SUITE 800, MCLEAN, VA, 22102, US

(21) Appl. No.: 09/732,927

(22) Filed: Dec. 11, 2000

(51) Int. Cl. A61N 5/00; G21G 5/00

(52) U.S. Cl. 250/492.2

(57) ABSTRACT

A method for manufacturing an object mask which is used in an electron beam proximity exposure apparatus comprising an electron beam source which emits a collimated electron beam, the object mask having an aperture which is arranged on a path of the electron beam, and a stage which holds and moves an object, wherein the object mask is arranged in proximity to a surface of the object and a pattern corresponding to the aperture of the object mask is exposed on the surface of the object with the electron beam having passed through the aperture, the method comprising the steps of: manufacturing a master mask having an aperture of a pattern identical with the object mask; and manufacturing a child mask by exposing an aperture pattern identical with the master mask by using the master mask in an electron beam proximity exposure method, wherein the child mask is used as the object mask. Thus, a manufacturing method of the masks for the electron beam proximity exposure at reduced costs is accomplished.

### BACKGROUND OF THE INVENTION

1. Field of the Invention

The present invention relates to a manufacturing method of a mask for an exposure apparatus used to expose fine patterns in a manufacturing process of semiconductor integrated circuits, etc., and more particularly to a manufacturing method of a mask used in an electron beam proximity exposure apparatus in which the mask having apertures corresponding to a pattern to be exposed is disposed in proximity to a surface of an object such as a semiconductor wafer, the mask is irradiated with an electron beam, and exposure of the pattern with the electron beam having passed through the apertures is thereby performed.

2. Description of the Related Art

Attempts are being made to enhance integration degrees of semiconductor integrated circuits and finer circuit patterns are desired. Presently, a limit of the finer circuit patterns is defined mainly by exposure apparatuses, and a stepper, which is an optical exposure apparatus, takes various measures such as a light source that emits rays having shorter wavelengths, a larger NA (numerical aperture) and a phase shift method. However, much finer circuit patterns involve various kinds of problems such as a rapid increase of manufacturing costs. New types of exposure apparatus such as an electron beam direct lithography apparatus and an X-ray exposure apparatus have been therefore developed, but there still remain many problems in terms of stability, productivity, cost, etc.

An electron beam proximity exposure system is conventionally under research and development, since the exposure principle thereof is simple, as “High Throughput Submicron Lithography with Electron Beam Proximity Printing” (H. Bohlen et al., Solid State Technology, September 1984, pp. 210-217) (hereinafter referred to as literature 1) exemplifies. However, it was thought that it was of no practical use since it was difficult to eliminate the proximity effect peculiar to the electron beam.

U.S. Pat. No. 5,831,272 (corresponding to Japanese Patent No. 2951947) and “Low energy electron-beam proximity projection lithography:Discovery of missing link” (Takao Utsumi, J. Vac. Sci. Technol. B 17(6), November/December 1999, pp. 2897-2902) disclose an electron beam proximity exposure apparatus that overcomes the above-mentioned problems and is usable for processing with very fine resolution at a mass production level.

FIG. 1 is a view showing a fundamental configuration to realize the electron beam proximity exposure apparatus disclosed in U.S. Pat. No. 5,831,272. Referring to this drawing, the electron beam proximity exposure apparatus disclosed in U.S. Pat. No. 5,831,272 will be briefly described. As shown in FIG. 1, in a column 10 are disposed an electron gun 12, which includes an electron beam source 14 emitting an electron beam 15, a shaping aperture 16, and a condenser lens 18 collimating the electron beam 15; scanning means 20, which includes a pair of main deflecting devices 22 and 24 and scans with the electron beam parallel to the optical axis; an object mask (hereinafter simply referred to as a mask) 30, which has apertures corresponding to an exposed pattern; and an object (a semiconductor wafer) 40, of which surface is coated with a resist layer. The mask 30 has a film 32 with the apertures formed at the center within a thick rim 34, and the object 40 is disposed so that the surface thereof is in proximity to the mask 30. In this state, when the electron beam is vertically applied to the mask, the electron beam passing through the mask's apertures is applied to the resist layer 42 on the surface of the object 40. The entire surface of the film 32 on the mask 30 is scanned by deflecting the electron beam 15 (A, B, and C in FIG. 1 denote the deflected beam toward three points) with the scanning means 20, so that all aperture patterns of the mask 30 are exposed. The scanning means 20 has subsidiary deflecting devices 51 and 52, which slightly lean the electron beam, and is used to position the mask 30 and the object 40 and to correct a difference between the exposure positions due to distortion of the mask and distortion of the object.

The mask for the electron beam proximity exposure is manufactured by exposing the pattern by a conventional electron beam exposure apparatus that can expose desired patterns. Such an apparatus takes an extremely long time for exposing patterns with high quality, and the costs of the masks are thereby increased.

Since a production amount of each kind of semiconductor integrated circuit is extremely large, one mask is used for multiple times of exposure. The mask for the electron beam proximity exposure is a stencil mask that has the apertures formed on the extremely thin film. Some deterioration can not be avoided because of the irradiating electron beam, and the mask is soiled first, so that the copied pattern is incorrect. In an extreme case, the film except the apertures disappears and white defects occur. Since the extent of deterioration depends on the irradiation amount of the electron beam, the durability of the mask is essential. In a case that insufficient durability of the mask limits numbers of exposure, many expensive masks need to be formed, which causes the problem of the high production costs of the semiconductor devices.

When the electron beam is applied on the resist layer formed on the surface of the object, a few molecules of the resist are scattered from the surface thereof, which result in the soil of the mask. Since the mask is actually disposed in proximity to the object, these molecules adhere to the surface of the mask and are charged-up, an electronic field is thereby produced, and an error with an irradiation of the electron beam is made as a result. Hence, the surface of the mask needs to be periodically cleaned by ozone-ashing or the like; however, the cleaning process also deteriorates the mask and causes the disadvantages of the low durability of the mask as described above and of the high manufacturing costs of the semiconductor devices.

### SUMMARY OF THE INVENTION

The present invention has been developed in view of the above-described circumstances, and has as its object the provision of a method for manufacturing masks for the electron beam proximity exposure at reduced costs.

The inventors of the present invention have directed their attention to the features that the electron beam proximity exposure apparatus is an actual-size exposure apparatus, an exposed pattern is identical with a pattern of the mask, and the electron beam proximity exposure apparatus can be used to copy the masks.

The method for manufacturing a mask for the electron beam proximity exposure according to the present invention is characterized in a method for manufacturing an object mask which is used in an electron beam proximity exposure apparatus comprising an electron beam source which emits a collimated electron beam, the object mask having an aperture which is arranged on a path of the electron beam, and a stage which holds and moves an object, wherein the object mask is arranged in proximity to a surface of the object and a pattern corresponding to the aperture of the object mask is exposed on the surface of the object with the electron beam having passed through the aperture, the method comprising the steps of: manufacturing a master mask having an aperture of a pattern identical with the object mask; and manufacturing a child mask by exposing an aperture pattern identical with the master mask by using the master mask in an electron beam proximity exposure method, wherein the child mask is used as the object mask.

The extremely large production amount and the insufficient durability of the mask may multiply a number of exposures with the master mask. In this case, to reduce a number of exposures with the master mask, it is possible that a further child mask (a grandchild mask) is formed by using the child mask copied from the master mask as the master mask, and the grandchild mask is used for practical exposing of the objects. The step of manufacturing the further child mask by using the child mask as the master mask is not limited to a single step, and more number of the steps can be performed (i.e., a great-grandchild mask may be formed by using the grandchild mask as the master mask, and so on).

To manufacture the mask for the electron beam proximity exposure, holes corresponding to an aperture pattern are formed, deeper than the thickness of film of a finished mask, on a surface of a thin plate, and the entire face of the mask is then etched from the other surface until the thickness reaches the thickness of the film of the finished mask. Accordingly, the master mask is exposed from the side facing the child mask, and the child mask is exposed from the side facing the object or the grandchild mask; then, the pattern formed by one exposure is right and left reversed from the previous pattern. When a number of manufacturing steps of the child mask using the master mask or the child mask is n times, n+1 times of exposures (i.e., reversals of the pattern) including the initial exposure for the master mask are performed; and hence, a pattern exposed on the master mask is set as a pattern right and left reversed from a pattern on the object when n+1 is an odd number, and the pattern exposed on the master mask is set as a pattern non-reversed from the pattern on the object when n+1 is an even number.

As described in the above, the mask for the electron beam proximity exposure is a very thin film, which is required to have an excellent flatness. Then, it is necessary to form a thin film on the surface of the film to apply a force in the direction of it shrinking so that a stress to tense the thin film portion is applied from the thick portion around the mask. However, the film for stressing causes a very small distortion on the aperture pattern, which results in a difference between the actual aperture pattern and a desired pattern.

As disclosed in U.S. Pat. No. 5,831,272, etc., the electron beam proximity exposure apparatus can correct a small distortion of the mask by adjusting a direction of the electron beam applied to the mask. Then, after the manufacturing of the master mask, it is preferable to measure an amount of distortion of the pattern thereof, and to perform the exposure of the child mask while correcting the amount of the distortion. Further, if the distortion produced on the child mask at the time a correct pattern is exposed can be estimated, it is preferable to correct the distortion estimated to be produced on the child mask as well as the substantial distortion produced on the master mask.

When the master mask and the child mask are made from the same material and have the same shape, the distortion amount mainly depends on the aperture pattern. In a case that the distortion to be produced on the child mask at the time the correct pattern is exposed is estimated to be equivalent to the distortion of the master mask, the distortion to be produced on the child mask can be corrected by determining that the correction amount is twice as much as the distortion of the master mask, so that the child mask without distortion can be obtained.

### BRIEF DESCRIPTION OF THE DRAWINGS

The nature of this invention, as well as other objects and advantages thereof, will be explained in the following with reference to the accompanying drawings, in which like reference characters designate the same or similar parts throughout the figures and wherein:

FIG. 1 is a view showing a fundamental configuration of an electron beam proximity exposure apparatus;

FIG. 2 is a diagram illustrating a fundamental concept according to the present invention;

FIG. 3 is a view showing a configuration of the electron beam proximity exposure apparatus used in an embodiment of the present invention;

FIG. 4(A) is a view showing a mask used in the electron beam proximity exposure apparatus, and FIG. 4(B) is a sectional view taken on line 4(B)-4(B) in FIG. 4(A);

FIGS. 5(A), 5(B), 5(C) and 5(D) are views illustrating a method of copying a child mask from a master mask according to the embodiment of the present invention;

FIG. 6 is a sectional view showing a structure of the mask to which a layer for stressing is applied used in the electron beam proximity exposure apparatus;

FIG. 7 is a view illustrating a method of exposing while correcting the mask's distortion in the electron beam proximity exposure apparatus;

FIG. 8 is a diagram explaining a correction of distortion produced on the mask; and

FIG. 9 is a diagram explaining a method of correcting also distortion that is estimated to be produced even when the correct pattern is exposed.

### DETAILED DESCRIPTION OF THE PREFERRED EMBODIMENTS

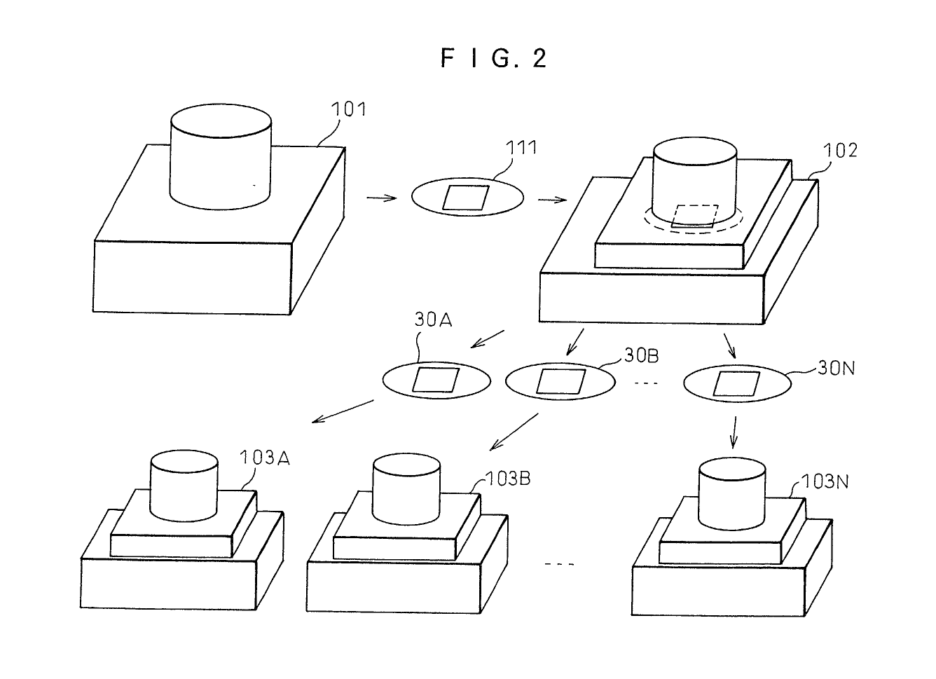

FIG. 2 is a view illustrating a fundamental concept according to the present invention.

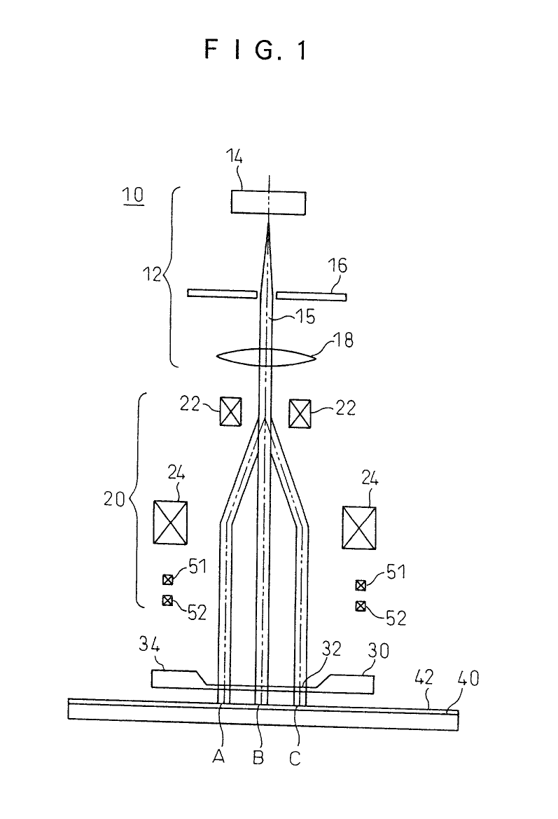

In FIG. 2, a reference number 101 denotes a conventional electron beam exposure apparatus that can expose any desired patterns. A master mask 111 is formed by exposing by the electron beam exposure apparatus 101 the same pattern as the one to be exposed on objects, and then by performing processes such as developing, etching, etc. A reference number 102 denotes an electron beam proximity exposure apparatus for copying masks, which has the similar configuration to the one disclosed in U.S. Pat. No. 5,831,272 as shown in FIG. 1. Exposures using the master mask 111 as the mask 30 in the electron beam proximity exposure apparatus 102 for copying masks are performed to manufacture many (N pieces) children masks 30A-30N. The children masks 30A-30N are mounted on electron beam proximity exposure apparatuses 103A-103N, respectively, and exposures onto the objects (semiconductor wafers) are performed. In this embodiment, there are a plurality of electron beam proximity exposure apparatuses for the exposure on the objects; however, in a case that there is a single electron beam proximity exposure apparatus, when a child mask is deteriorated and becomes unusable, another child mask can be used.

FIG. 3 is a view showing a configuration of the electron beam proximity exposure apparatus according to the first embodiment of the present invention. This apparatus is used for exposures on the child masks and the objects. Since the fundamental configuration is similar to the one shown in FIG. 1 and the one disclosed in the above literature 1, the same function parts with FIG. 1 are denoted with the same reference numbers.

As shown in FIG. 3, in an electron optical column 10 are disposed an electron gun 14, which emits electron beam 15, a condenser lens 18, which collimates the electron beam 15, a main deflecting device 20 and a subsidiary deflecting device 50. Although shown as a single deflecting device in FIG. 3, each of the main deflecting device 20 and the subsidiary deflecting device 50 is actually configured in two stages as shown in FIG. 1. In a vacuum object chamber 8 are disposed a mask stage 36, which holds and moves a mask 30, and a stage 131, which holds and moves a substrate 121 corresponding to a material of the child mask or the object.

In FIG. 3, a state is shown where an applied position of the electron beam 15 on the mask 30 is changed by the main deflecting device 20. As illustrated, even when the main deflecting device 20 changes the applied position, the electron beam 15 is substantially vertically applied to the mask 30.

In the above, the electron beam proximity exposure apparatus according to the first embodiment of the present invention is described, the fundamental configuration is similar to the one disclosed in the literature 1 and U.S. Pat. No. 5,831,27,2, and hence, further detailed explanation is omitted and only different points are described in the below.

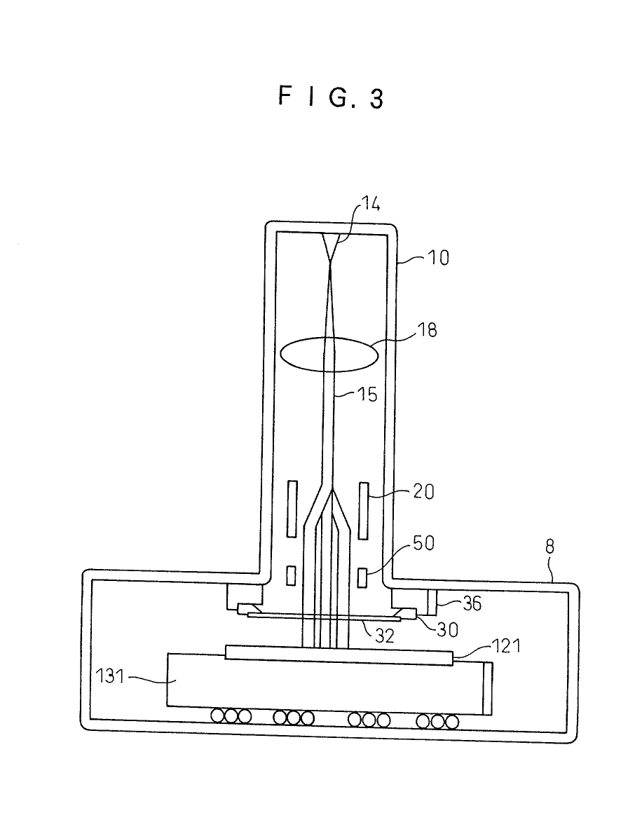

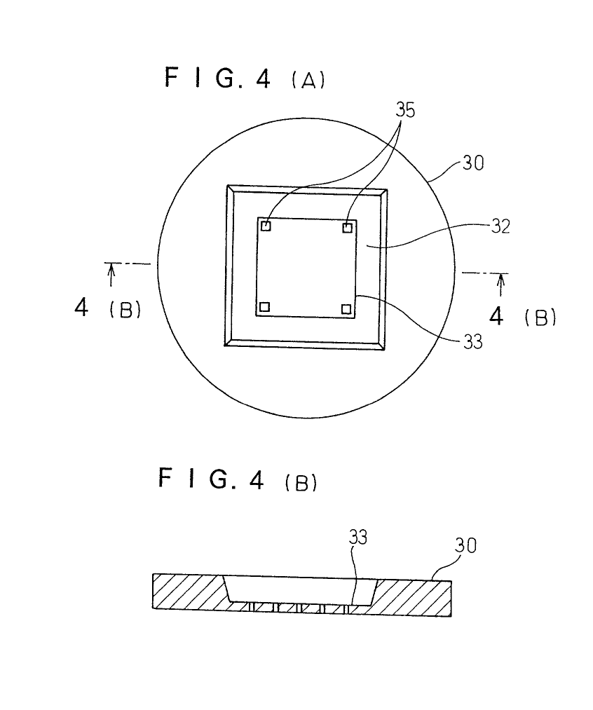

FIGS. 4(A) and 4(B) are a perspective view and a sectional view, respectively, of the mask. The master mask 111 and the child mask 30 have the same shape and are made from the same material. As shown in FIGS. 4(A) and 4(B), the mask 30 is a thin plate member with a thickness of a few millimeters, for example, and center portion denoted with a reference number 32 is processed in the thickness of a few micrometers, which an aperture pattern is formed in a portion denoted with a reference number 33 within. A reference number 35 denotes a mark for determining a mask's position.

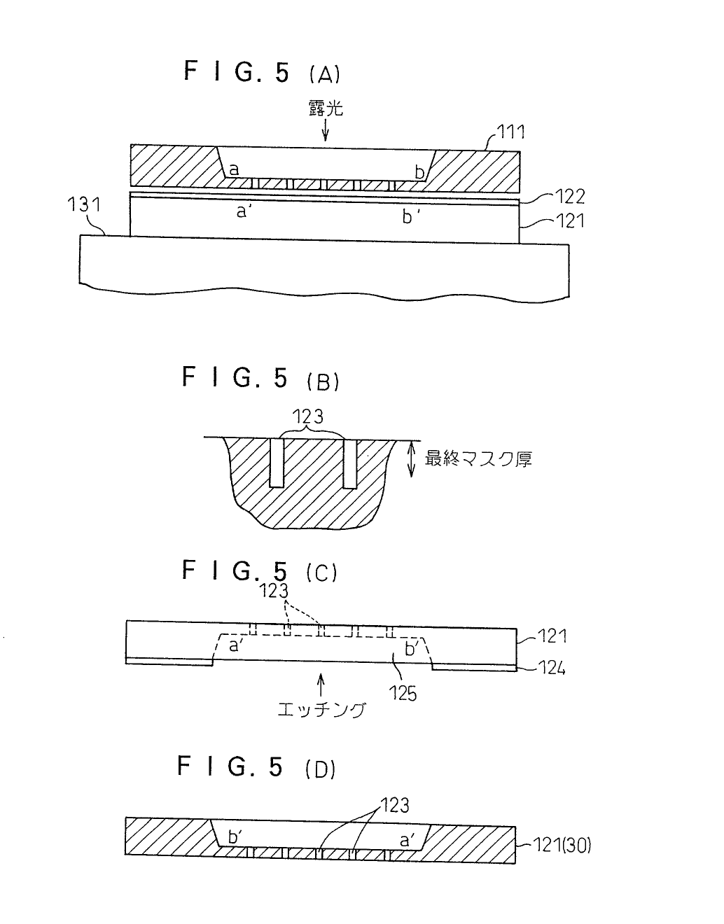

FIGS. 5(A), 5(B), 5(C) and 5(D) are explanatory views of the manufacturing process according to the embodiment of the present invention to copy the child masks from the master mask. In this embodiment, the same apparatus is used for exposure as the electron beam proximity exposure apparatus shown in FIG. 1.

As shown in FIG. 5(A), the master mask 111 is mounted as the mask 30. The master mask 111 can be formed by any method, in which for example, the desired pattern is exposed on the resist applied on the substrate of the mask by the conventional electron beam exposure apparatus, and processing such as developing and etching is performed. Next, a substrate 121 for the child mask of which first surface is coated with a resist layer 122 is fixed on the stage 131 of the electron beam proximity exposure apparatus and is arranged in proximity to the master mask 111. In this state, the electron beam is applied to expose the pattern corresponding to the aperture pattern of the master mask 111.

Then, the resist of the aperture parts is removed by developing the resist, and holes 123 are formed as shown in FIG. 5(B) by etching the first surface. The holes correspond to the aperture pattern and are made a little deeper than the finished mask's thickness (the thickness of the portion denoted with the reference number 32 in FIG. 4(A)).

Next, as shown in FIG. 5(C), a resist layer 124 is formed on a portion of a second surface except for a portion corresponding to the portion denoted with the reference number 32 in FIG. 4(A), and the second surface is etched so as to make the portion denoted with the reference number 125 has the thickness of the finished mask. Thereby, the aperture parts 123 in FIG. 5(B) are perforated and the aperture pattern is formed. The child mask is thus formed as shown in FIG. 5(D).

As shown in FIG. 5(A), portions denoted with a and b of the master mask 111 correspond to portions denoted with a′ and b′ of the child mask 121, respectively. In a state where the child mask is used, since the child mask is inverted, a′ and b′ are with right and left reversed as shown in FIG. 5(D). By comparison between FIG. 5(A) and FIG. 5(D), it is seen that the pattern of the master mask and the pattern of the child mask are equivalent but are with right and left reversed to each other. Accordingly, this is required to be considered for determining a direction of the pattern when the pattern is exposed on the master mask. For instance, in a case that also the master mask is formed as illustrated in FIGS. 5(A)-5(D) by forming a resist layer on the first surface of the substrate of the master mask, exposing the pattern on the resist layer, forming holes corresponding to the apertures, and etching the second surface up to the finished mask's thickness; the master mask's pattern is with right and left reversed. The child mask's pattern copied from the master mask is also with right and left reversed, and the child mask's pattern is hence identical with the pattern that has been exposed on the master mask's substrate at first, which is not reversed. Therefore, the non-reversed pattern, which is identical with the pattern to be exposed on the objects, is exposed to obtain the pattern for the master mask.

As described above, according to the embodiment of the present invention, the master mask is used to expose the child mask's pattern, and the child mask is used for the exposure on the objects. However, in order to make a frequency of the use of the master mask as little as possible, the further children masks can be formed as follows: the first generation children masks, which are produced by exposure with the master mask, are used as master masks to produce the second generation children masks (grandchildren masks), and the second generation children masks are then used as master masks to produce the third generation children masks (great-grandchildren masks) and so on; then the further children masks are used for the exposure on the objects. Consequently, even if a number of exposures of the mask is extremely large, a number of the exposures using the master mask can be kept small, and risk of damaging the master mask is thus extremely reduced. In this case, since the pattern becomes with right and left reversed whenever the exposures of the master mask and the child mask are performed, when a number of forming process of the child mask from the master mask or the child mask is n times, n+1 times of exposures including the initial exposure for the master mask, i.e., reversals of the pattern are performed. Hence, when n+1 is an odd number, the pattern to be exposed on the master mask is set as a pattern that is right and left reversed from the pattern to be exposed on the object; and when n+1 is an even number, the pattern to be exposed on the master mask is set as a pattern that is not reversed from the pattern to be exposed on the object.

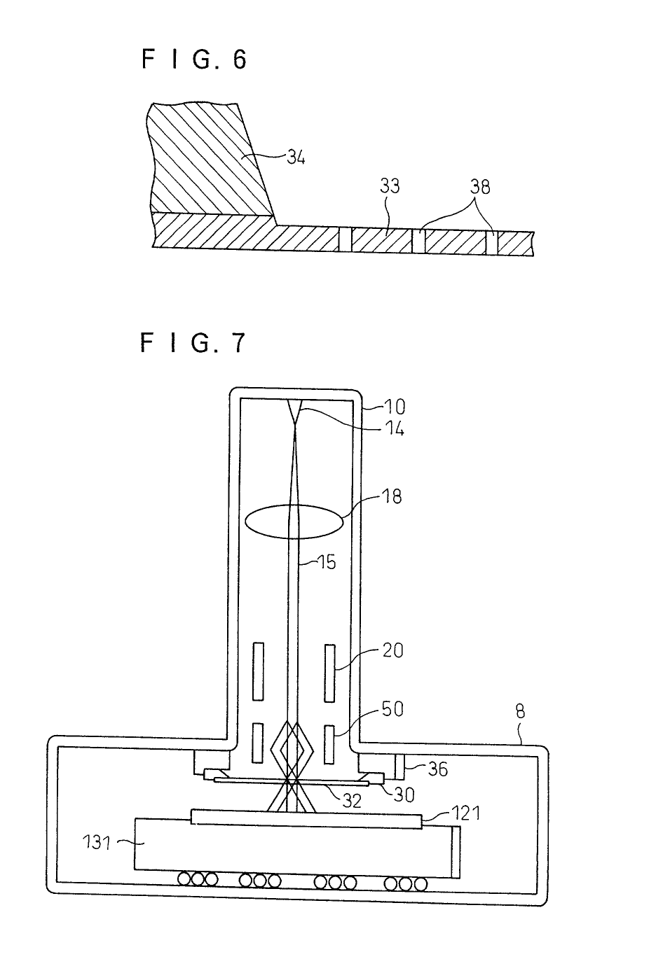

The mask for the electron beam proximity exposure comprises an extremely thin film with its size of tens by tens millimeters and thickness of a few micrometers or less than one micrometer, and the film is required to have an excellent flatness. Then, as illustrated in FIG. 6, the film 33 of the mask is made of material that applies a force on the surface of the mask's film 33 in the direction of it shrinking. For example, Si (wafer) is nitrified to form a film portion of SiN. Thereby, the stress tenses the film portion 33 from the thick portion 34 around the mask, and the excellent flatness is thus achieved. However, the film 33 has apertures 38 of which pattern is partially different, so that the stress is partially varied and it causes distortion on the film 33 of the mask.

The above-described literature 1 and U.S. Pat. No. 5,831,272 disclose a technique in the electron beam proximity exposure apparatus to correct the distortion on the mask by changing a direction of the electron beam applied to the mask. This technique is utilized to form the mask with the reduced distortion in the processing method of the mask according to the present embodiment.

FIG. 7 is a view showing the technique to correct the distortion on the mask disclosed in the above references.

When a deflecting amount of the main deflecting device 20 is changed so as to change the applied position of the electron beam 15 on the mask 30, the electron beam 15 is substantially vertically applied to the mask 30 as shown in FIG. 3.

In contrast, as shown in FIG. 7, when the subsidiary deflecting device 50 changes an incident angle of the electron beam 15 onto the mask 30, the electron beam 15 falls on the same position on the mask 30 while the incident angle is changed. As the incident angle is changed, the applied position on the substrate 121 is changed in spite of the electron beam having passed through the same position of the mask. The changing amount is a product of the incident angle and the distance between the mask and the substrate. Hence, the distortion amount of the mask 30 is determined in advance, the incident angle is set so that the changing amount of the applied position according to the incident angle is equivalent and in the compensating direction to the distortion amount, and the distortion of the mask can be thus corrected.

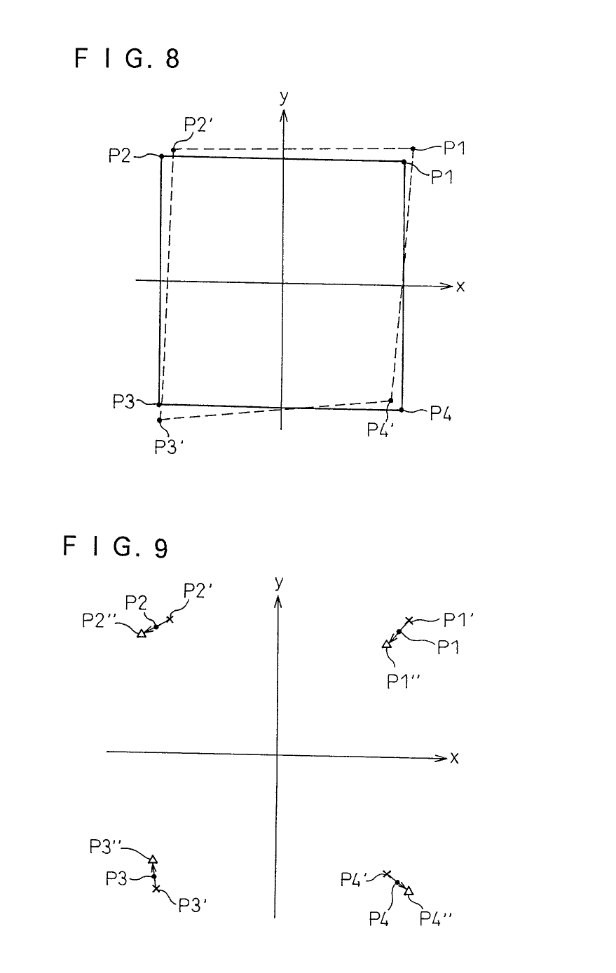

The smaller electron beam scanning the mask can theoretically correct any distortion; however, it is preferable that the electron beam has a certain size to satisfy the throughput, and in such a case, a rather large distortion cannot be corrected. Also, although the distortion can be corrected even if it is non-linear, an example is described where the correction is performed while approximating the distortion to be linear as shown in FIG. 8 in order to simplify the control of the subsidiary deflecting device.

As illustrated in FIG. 8, points P1-P4 are respectively exposed at desired positions while the master mask is exposed. However, as a result of forming of holes corresponding to the apertures and processing of the film portion, suppose that the points P1, P2, P3 and P4 have been formed at the points P1′, P2′, P3′ and P4′, respectively, in the master mask that has been actually manufactured. This is considered as the original ideal XY coordinates are linear-transformed into actually distorted xy coordinates. In order to correct the distorted xy coordinates to the original ideal XY coordinates, a linear transformation is performed by the following transformational functions:

X=a₁+a₂·x+a₃·y+a₄·xy; and

Y=b₁+b₂·x+b₃·y+b₄·xy,

where a₁-a₄ and b₁-b₄ are correction factors for the mask's distortion.

The correction factors a₁-a₄ and b₁-b₄ are obtained by substituting the coordinates of P1′-P4′ and the coordinates of P1-P4 into the above transformational functions.

The incident angle is determined according to the correction amount at each point on the mask that is calculated from the above transformational functions, the deflecting amount of the subsidiary deflector is determined, and the exposure is then performed, so that the pattern without distortion can be exposed on the child mask's substrate even if the master mask has been distorted.

However, although the pattern without distortion is exposed on the child mask's substrate as described in the above, distortion is expected to be produced in processing into the child mask. As described above, since the distortion is mainly produced by partial differences between the aperture patterns of the film and the stressing film, the distortion is expected to have reproducibility. Hence, a child mask is once formed by exposing the aperture pattern without distortion, and distortion produced on the child mask is measured, so that the distortion produced on the child mask is analyzed. Then, the distortion estimated to be produced on the child mask as well as the distortion of the master mask are corrected together at the time of exposing the child mask using the master mask, whereby, the child mask finally obtained can have the aperture pattern without distortion.

In a case that the master mask and the child mask are made from the same material and have the same shape, the distortion that is equivalent to the distortion of the master mask is expected to be produced on the child mask. Hence, as illustrated in FIG. 9, the coordinates of P1′-P4′ are transformed into the coordinates of P1″-P4″ by doubling the correction amount for example, rather than transforming the coordinates of P1′-P4′ into the coordinates of P1-P4. Thereby, although the pattern distorted in the inverse direction is exposed on the child mask's substrate, this distortion is compensated with the distortion produced in a process of finishing the child mask, and the child mask having the ideal pattern without distortion is obtained as a result.

As described in the above, according to the present invention, after producing a master mask only once, masks to be used for practical exposing of the objects can be easily manufactured at the reduced costs, and even though the mask's durability is low, it less influences manufacturing costs of semiconductor devices. Furthermore, the masks are copied form the master mask by the electron beam proximity exposure method, which can correct distortion, so that the masks finally obtained are extremely precise masks with less distortion than the master mask.

It should be understood, however, that there is no intention to limit the invention to the specific forms disclosed, but on the contrary, the invention is to cover all modifications, alternate constructions and equivalents falling within the spirit and scope of the invention as expressed in the appended claims.

What is claimed is:

1. A method for manufacturing an object mask which is used in an electron beam proximity exposure apparatus comprising an electron beam source which emits a collimated electron beam, the object mask having an aperture which is arranged on a path of the electron beam, and a stage which holds and moves an object, wherein the object mask is arranged in proximity to a surface of the object and a pattern corresponding to the aperture of the object mask is exposed on the surface of the object with the electron beam having passed through the aperture, the method comprising the steps of:
manufacturing a master mask having an aperture of a pattern identical with the object mask; and
manufacturing a child mask by exposing an aperture pattern identical with the master mask by using the master mask in an electron beam proximity exposure method,
wherein the child mask is used as the object mask.

2. The method as defined in claim 1, wherein:
the master mask is exposed from a side facing the child mask;
the child mask is exposed from a side facing the object; and
a pattern exposed on the master mask is a pattern non-reversed from a pattern on the object.

3. The method as defined in claim 1, further comprising at least one step of manufacturing a further child mask by using the child mask as the master mask in the electron beam proximity exposure method.

4. The method as defined in claim 3, wherein:
the master mask is exposed from a side facing the child mask;
the child mask is exposed from a side facing one of the object and the further child mask; and
a number of the steps of manufacturing the child mask is n times, a pattern exposed on the master mask is a pattern right and left reversed from a pattern on the object when n+1 is an odd number, and the pattern exposed on the master mask is a pattern non-reversed from the pattern on the object when n+1 is an even number.

5. The method as defined in claim 1, wherein:
distortion of the master mask with respect to a desired pattern is determined after the manufacturing thereof; and
when an exposure is performed using the master mask, an application direction of the electron beam is changed so as to correct the determined distortion.

6. The method as defined in claim 5, wherein when the child mask is exposed using the master mask, the application direction of the electron beam is changed so as to correct the determined distortion as well as distortion estimated to be produced on the child mask.

7. The method as defined in claim 6, wherein:
the master mask and the child mask are made from the same material and have the same shape;
the distortion estimated to be produced on the child mask is equivalent to the distortion determined on the master mask; and
when the child mask is exposed using the master mask, the application direction of the electron beam is changed so as to correct a double amount of the determined distortion of the master mask.

8. A mask manufactured in accordance with the method of claim 1.

9. A mask manufactured in accordance with the method of claim 2.

10. A mask manufactured in accordance with the method of claim 3.

11. A mask manufactured in accordance with the method of claim 4.

12. A mask manufactured in accordance with the method of claim 5.

13. A mask manufactured in accordance with the method of claim 6.

14. A mask manufactured in accordance with the method of claim 7.
